(19) United States
(12) Patent Application Publication
(10) Pub. No.: US 2013/0083177 A1
(43) Pub. Date: Apr. 4, 2013

(54) SYSTEM AND METHOD FOR MOSAICKING ENDOSCOPE IMAGES CAPTURED FROM WITHIN A CAVITY

(71) Applicants: Jason J. CORSO, Buffalo, NY (US); Michael SMITH, Santa Monica, CA (US); Marcus O. FILIPOVICH, Venice, CA (US)

(72) Inventors: Jason J. CORSO, Buffalo, NY (US); Michael SMITH, Santa Monica, CA (US); Marcus O. FILIPOVICH, Venice, CA (US)

(73) Assignee: Ikona Medical Corporation, Venice, CA (US)

(21) Appl. No.: 13/491,027

(22) Filed: Jun. 7, 2012

Related U.S. Application Data
(63) Continuation of application No. 12/347,855, filed on Dec. 31, 2008, now abandoned.

(51) Int. Cl. A61B 1/05

(52) U.S. Cl. CPC A61B 1/05
USPC 348/65

(57) ABSTRACT

Systems and methods for capturing and mosaicking images of one or more surfaces of a collapsed cavity are described. One embodiment includes capturing images using an endoscope, where the optics of the endoscope are radially symmetrical, locating the optical center and dewarping each of the captured images by mapping the image from polar coordinates centered on the optical center of the image to rectangular coordinates, discarding portions of each dewarped image to create high clarity dewarped images, estimating the motion of the endoscope with respect to the interior surface of the cavity that occurred between successive high clarity dewarped images, registering the high clarity dewarped images with respect to each other using the estimates of motion, and combining the registered high clarity dewarped images to create at least one mosaic.

### RELATED APPLICATION

This application is a continuation of U.S. application Ser. No. 12/347,855 filed Dec. 31, 2008, the disclosure of which is incorporated herein by reference.

### STATEMENT OF FEDERAL SUPPORT

The U.S. Government has certain rights in this invention pursuant to Grant No. HD054974 awarded by the National Institute of Child Health and Human Development.

### BACKGROUND

Endoscopy is a minimally invasive diagnostic medical procedure that can be used to assess the interior surfaces of a cavity. Many endoscopes include the capability of capturing images. When the endoscope captures images of the interior surface of a cavity, the images can be mosaiced together to provide a map of the interior surface of the cavity. The manner in which the images are mosaiced typically depends upon the type of endoscope used and the cavity being imaged.

Many endoscopic procedures involve insufflating and imaging a normally collapsed space. For example, a collapsed cavity. A collapsed cavity is a cavity in which the walls of the cavity are in contact with each other. The cavity itself can be any shape including a lumen, a cavity that is defined by two or more walls, or a network of cavities. Insufflation or inflation endoscopic techniques, use a distending media to insufflate or inflate the collapsed cavity during imaging. In many instances, capturing images without insufflating the cavity can be more comfortable and safer for a patient. The endoscope described in U.S. patent application Ser. No. 10/785,802 entitled “Method and Devices for Imaging and Biopsy” to Wallace et al., the disclosure of which is incorporated by reference herein in its entirety, describes an endoscope that can be used to image the uninsufflated uterus of a patient. Instead of insufflating the cavity, Wallace et al. describe the use of a contact endoscope in which images are taken of the inside lining of a body cavity that is coapted around the tip of the endoscope.

### SUMMARY OF THE INVENTION

Systems and methods for capturing and mosaicking images of one or more surfaces of a collapsed cavity are described. One embodiment includes capturing images using an endoscope, where the optics of the endoscope are radially symmetrical, locating the optical center of each of the captured images, dewarping each of the captured images by mapping the image from polar coordinates centered on the optical center of the image to rectangular coordinates, discarding portions of each dewarped image to create high clarity dewarped images, estimating the motion of the endoscope with respect to the interior surface of the cavity that occurred between successive high clarity dewarped images, registering the high clarity dewarped images with respect to each other using the estimates of motion, and combining the registered high clarity dewarped images to create at least one mosaic.

In a further embodiment, the optical center is located by comparing the captured image to a template image.

In another embodiment, the optical center is located by locating the translation between the captured image and the template image that results in the smallest sum of absolute differences.

In a still further embodiment, a large range of possible translations are considered in locating the optical center of a first captured image, and a small range of possible translations are considered relative to the location of the optical center of the first captured image in locating the optical center of a second captured image.

In still another embodiment, the endoscope includes a tip through which images are captured and the tip includes fiducial markings that assist in the location of the optical center of captured images.

In a yet further embodiment, discarding portions of each dewarped image that possess insufficient image clarity to create high clarity dewarped images comprises discarding a predetermined portion of each dewarped image.

In yet another embodiment, discarding portions of each dewarped image that possess insufficient image clarity to create high clarity dewarped images includes performing blur detection on each image, and discarding at least one region of the image possessing blurriness exceeding a predetermined threshold.

A further embodiment again also includes adjusting the brightness of pixels to account for variations in the illumination of the imaged surface.

Another embodiment again also includes compensating for pixels within the captured images that are the result of known defects in the endoscope.

In a further additional embodiment, estimating the motion of the endoscope with respect to the interior surface of the cavity that occurred between successive high clarity dewarped images comprises determining a motion vector at which the square of the differences between successive captured images is a minimum.

In another additional embodiment, determining the motion vector at which the square of the differences between successive captured images is a minimum includes creating a Gaussian pyramid for each image, and using the motion vector at which the square of the differences between images of corresponding lower resolution in the Gaussian pyramids is a minimum to determine the motion vector at which the square of the differences between images of corresponding higher resolution images in the Gaussian pyramids is a minimum, until the motion vector at which the square of the differences between the two captured images is a minimum is determined.

A still yet further embodiment includes performing multiple passes over the captured images to improve the accuracy of the estimation of the motion of the endoscope with respect to the interior surface of the cavity compared to the initial estimate determined by comparing successive high clarity dewarped images, and performing multiple passes to register successive images with respect to each other to reduce registration errors accumulated during initial sequential registration.

In still yet another embodiment, images are captured at a frame rate chosen so that the motion that occurs between the captured images is sufficiently small for the high clarity dewarped images to overlap.

In a still further embodiment again, the captured images are color images, the system generates mosaics in real time, and processing latency is reduced by converting the captured images from color to grayscale and locating the optical center of the grayscale images.

In still another embodiment again, processing latency is reduced by dewarping grayscale images and performing motion estimation using grayscale images.

A still further additional embodiment includes performing image segmentation to identify segments of the image corresponding to different surfaces of the cavity and combining the image segments corresponding to different surfaces of the cavity to form separate mosaics of each of the different surfaces of the cavity.

In still another additional embodiment, performing image segmentation further includes locating at least the two darkest columns in the high clarity dewarped images.

A yet further embodiment again includes limiting the rotation of the endoscope and assuming the two darkest columns are constrained to be located within defined regions of the high clarity dewarped images.

In yet another embodiment again the defined regions correspond to the two halves of the field of view of the endoscope.

A yet further additional embodiment further includes using boundaries between groups of aligned motion vectors of blocks of pixels within the image to identify segments of the image corresponding to different surfaces of the cavity.

In yet another additional embodiment combining the registered high clarity dewarped images to create at least one mosaic comprises performing alpha blending using overlapping portions of high clarity dewarped images.

In a further additional embodiment again, the weighting applied during alpha blending is determined based upon the relative clarity of each of the overlapping portions of the high clarity dewarped images.

In another additional embodiment again, overlapping portions of the high clarity dewarped images are combined to create hyper-resolution image information.

Another further embodiment includes capturing images using an endoscope having a tip including fiducial markings, locating the optical center of each of the captured images using the fiducial markings on the endoscope tip, dewarping each of the captured images, discarding portions of each dewarped image to create high clarity dewarped images, estimating the motion of the endoscope with respect to the interior surface of the cavity that occurred between successive high clarity dewarped images, registering the high clarity dewarped images with respect to each other using the estimates of motion, and combining the registered high clarity dewarped images to create at least one mosaic.

Yet another further embodiment also includes performing multiple passes over the captured images to improve the accuracy of the estimation of the motion of the endoscope with respect to the interior surface of the cavity compared to the initial estimate determined by comparing successive high clarity dewarped images, and performing multiple passes to register successive images with respect to each other to reduce registration errors accumulated during initial sequential registration.

### BRIEF DESCRIPTION OF THE DRAWINGS

FIG. 1 is a semi-schematic diagram of an endoscopic image processing system in accordance with an embodiment of the invention.

FIG. 2 is a cross sectional view along a long axis of an endoscope inserted in a naturally collapsed or uninsufflated cavity.

FIG. 3a is a cross sectional view of the endoscope shown in FIG. 2 along line 22 when the endoscope is inserted within a collapsed lumen.

FIG. 3b is an image captured using an endoscope similar to the endoscope shown in FIG. 3a with a collapsed lumen test target.

FIG. 4a is a cross section view of the endoscope shown in FIG. 2 along line 22 when the endoscope is inserted within a collapsed cavity.

FIG. 4b is an image captured using an endoscope similar to the endoscope shown in FIG. 4a within a collapsed cavity.

FIG. 5 is a flow chart showing a process for constructing a mosaic of images captured using an endoscope in accordance with an embodiment of the invention.

FIG. 6 is a flow chart showing a process for acquiring image information from an endoscope that can be used in combination with the process 50 shown in FIG. 5 in accordance with an embodiment of the invention.

FIG. 7 is a process for dewarping an endoscope image captured within a collapsed lumen that can be used in combination with the process 50 shown in FIG. 5 in accordance with an embodiment of the invention.

FIG. 7a is an image generated by dewarping the captured endoscope image shown in FIG. 3b in accordance with an embodiment of the invention.

FIG. 8 is a process for mosaicking dewarped images captured within a collapsed lumen that can be used in combination with the process 50 shown in FIG. 5 in accordance with an embodiment of the invention.

FIG. 9 is a process for mosaicking dewarped images captured within a collapsed cavity that can be used in combination with the process 50 shown in FIG. 5 in accordance with an embodiment of the invention.

FIG. 9a is an image generated by dewarping the endoscope image shown in FIG. 4b in accordance with an embodiment of the invention.

FIGS. 10a and 10b conceptually illustrate rotation of an endoscope tip in the xz-plane.

FIGS. 11a and 11b conceptually illustrate rotation of an endoscope tip in the xy-plane.

FIGS. 12a and 12b conceptually illustrate translation of an endoscope tip along the z-axis.

FIGS. 13a and 13b conceptually illustrate translation of an endoscope tip in the xy-plane.

FIGS. 14a-14k conceptually illustrate the manner in which the anterior and posterior plane images are identified from a dewarped captured image based upon different locations of the two darkest columns of the dewarped captured image in accordance with an embodiment of the invention.

### DETAILED DESCRIPTION OF THE INVENTION

Turning now to the drawings, systems and methods for mosaicking images of the interior surface of a cavity coapted around the tip of an endoscope to produce a map of the interior surface of the cavity are described. In several embodiments, the tip of the endoscope is inserted within a cavity that is a lumen. The cavity can be completely collapsed or coapted, or the cavity can be partially collapsed or collapsed in some regions and not others. The endoscope itself can take any of a variety of forms including a rigid endoscope, a flexible endoscope or a capsule endoscope. In many embodiments, the mosaicking process may involve capturing each image, dewarping the image, performing motion estimation, and constructing one or more mosaics.

When an endoscope captures images inside a collapsed cavity, portions of the image can correspond to different surfaces or walls of the cavity. In several embodiments, the portions of the image corresponding to different surfaces of the cavity are identified using a segmentation process prior to performing motion estimation. In a number of embodiments, segmentation is performed by identifying the darkest regions of the image and tracking rotation to ensure that the segmentation process correctly assigns the segments of an image to the appropriate mosaics of the different surfaces of the cavity.

Motion estimation within a cavity is complex, because the tip of the endoscope is often free to move in three dimensions and has freedom of rotation around multiple axes of rotation. In a number of embodiments, motion estimation is performed by building a Gaussian pyramid of images based upon the dewarped time t captured image and a Gaussian pyramid of images based upon the dewarped time t−1 captured image and then using the Gaussian pyramids to perform motion tracking or motion estimation. Once motion estimation has been performed, the portions of the captured image corresponding to difference surfaces of the cavity, such as the anterior and posterior walls, are added to the map of the interior surface of the cavity. In several embodiments, the map includes separate anterior and posterior mosaics. In many embodiments, signal processing is performed using grayscale images to reduce the processing required to perform dewarping, anterior/posterior wall segmentation, and/or motion tracking. In several embodiments, the reduction in processing achieved through the use of grayscale images enables the creation of a color map of the interior surface of a cavity in real time using an off-the-shelf portable computer system.

### System Architecture

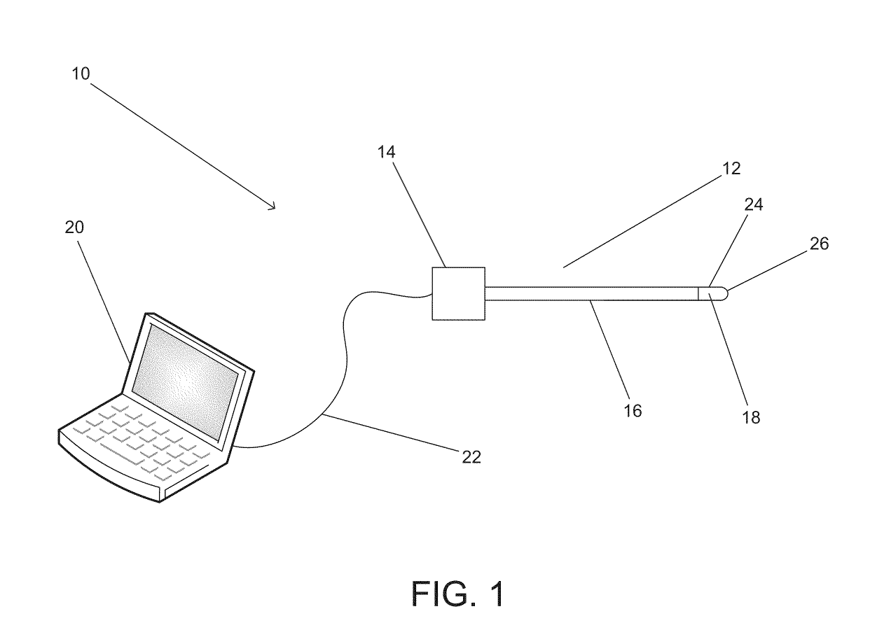

An endoscopic image processing system in accordance with an embodiment of the invention is shown in FIG. 1. The system 10 includes an endoscope 12. In the illustrated embodiment, the endoscope includes a body section 14 from which an imaging channel 16 extends. A tip 18 is fixed to the end of the imaging channel. At least a portion of the tip is transparent to enable the illumination of the interior of a cavity and the capturing of images of the interior surface of the cavity through the tip. The endoscope 12 is configured to communicate with a computer 20 via a cable 22. In the illustrated embodiment, the tip 18 has a cylindrical imaging surface 24 and a rounded insertion surface 26.

The body section 14 contains a camera or a connection to a camera and a light source or a connection to a light source. The light source can direct light through a coaxial illumination channel surrounding the imaging channel 16 to illuminate the interior surface of a cavity through the tip 18. The tip directs light reflected from the interior surface of the cavity down the imaging channel 16 to enable the capturing of images using the camera (not shown). The camera may comprise, but is not limited to monochrome imagers, color imagers, single CCD cameras, multi-CCD cameras, thermal imagers, or hyperspectral imagers. The endoscope camera can transmit captured images to the computer 20 via the cable 22. In one embodiment, the cable and the manner in which the endoscope and the computer communicate conforms to the USB 2.0 standard. In other embodiments, other wired and/or wireless communication standards can be used to exchange information between the endoscope and the computer.

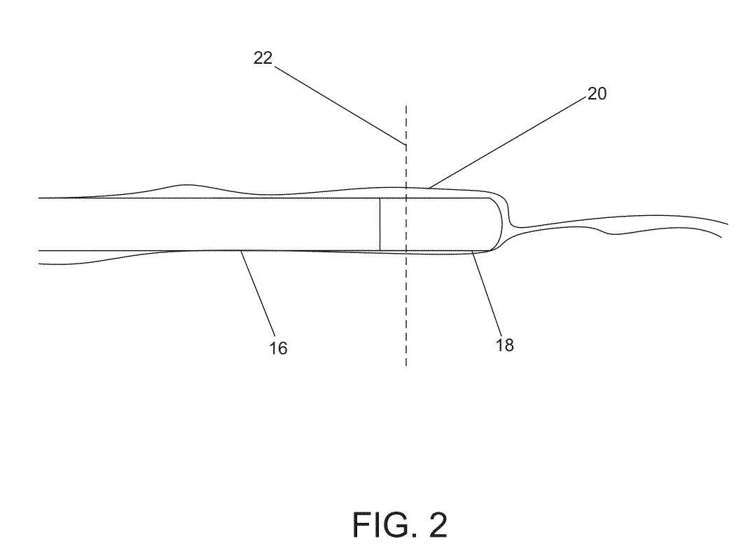

Due to the shape of the tip, the endoscope is able to capture images of the interior surface of a collapsed cavity coapted around the tip. A cavity coapted around a tip of an endoscope in accordance with an embodiment of the invention is shown in FIG. 2. A portion of the imaging channel 16 and the tip 18 are inserted within an uninsufflated cavity. The endoscope is able to capture images of portions of the interior surface of the cavity 20 coapted around the tip of the endoscope. In addition, the optics of the tip are radially symmetric. As is discussed further below, the radial symmetry of the optical tip enables dewarping using a one dimensional dewarping function. In a number of embodiments, the radially symmetric tip includes a cylindrical imaging section that enables imaging of an increased surface area of the cavity without increasing the width of the endoscope tip. In other embodiments, the optics of the tip are not radially symmetrical and a multi-dimensional dewarping function can be applied to dewarp the captured images.

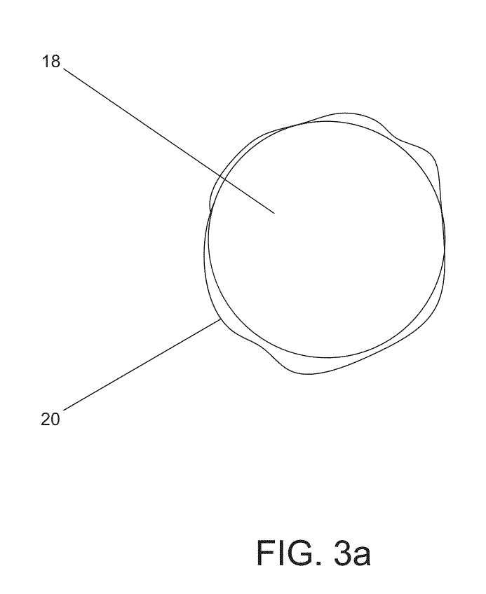

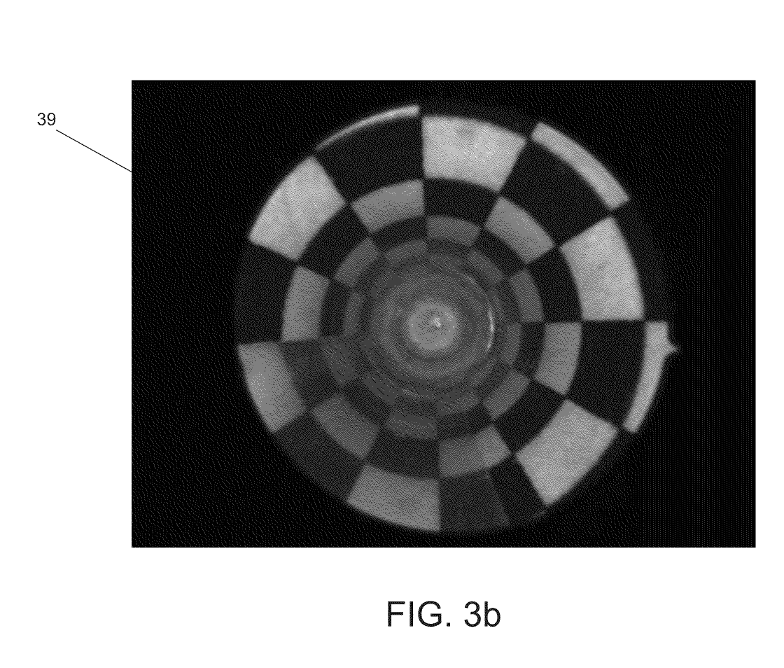

A cross section taken along the section 22 when the endoscope is within a collapsed cavity, where the cavity is a lumen coapted around the endoscope, is shown in FIG. 3a. The interior surface of the lumen coapts around at least a portion of the tip of the endoscope. The endoscope can rotate within the lumen and advance or retreat within the lumen. An example of an image captured from within a lumen is shown in FIG. 3b. The example image 39 is of a test lumen possessing a grid pattern on its interior surface.

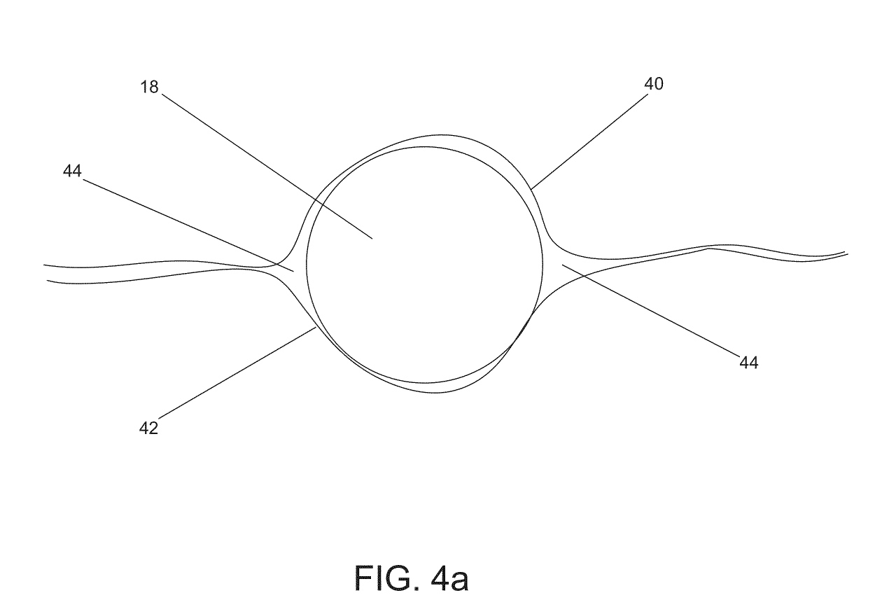

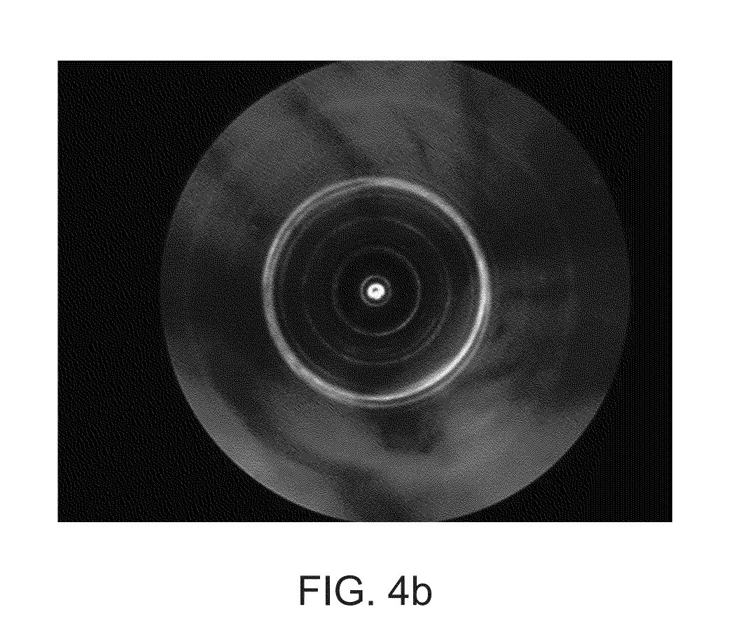

A cross section taken along the section 22 shown in FIG. 2 when the endoscope is located within a collapsed cavity is shown in FIG. 4a. A first wall 40 and a second wall 42 of the cavity are shown coapted around the tip 18 of the endoscope. In many instances, walls coapted around the tip of an endoscope only contact a portion of the tip and small gaps 44 exist between the tip and the walls, including locations at which the walls contact.

An image captured from within a collapsed cavity using an endoscope similar to the endoscope shown in FIG. 1 is shown in FIG. 4b. The gaps between the walls can be identified as regions of darkness. Although the example shown in FIG. 4a shows a cavity in which two walls coapt around the tip of the endoscope, embodiments of the invention can be used in cavities where more than two surfaces of the cavity coapt around the tip of the endoscope. For example, folds in the interior surface of a cavity can create images in which three or more surfaces are visible.

Although specific embodiments of endoscopes are described above, the image processing techniques described below can be used to mosaic images from a variety of different endoscopes, including flexible endoscopes and endoscopes that are completely contained within the cavity during the capture of images, such as capsule endoscopes.

### Image Processing

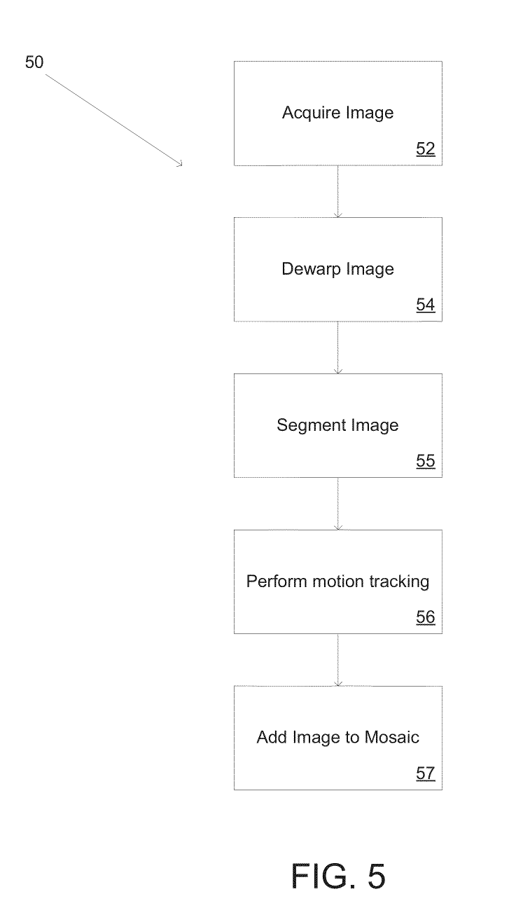

A process for mosaicking images captured using an endoscope within a collapsed cavity in accordance with an embodiment of the invention is shown in FIG. 5. The process 50 includes acquiring (52) an image, dewarping (54) the image, segmenting (55) the dewarped image, motion tracking (56) using the segmented images and adding (57) the segmented images to a mosaic. The specific nature of each step depends upon the components of the endoscope and the nature of the cavity. The ordering of the steps in FIG. 5 are given in a preferred order. However, certain embodiments are possible using a different ordering of the steps (i.e. the embodiment performs motion tracking 56 prior to dewarping 54). In instances where the interior surface of the cavity is a lumen coapted around the tip of the endoscope, segmentation may not be required. Various embodiments of processes used during image processing in accordance with embodiments of the invention are discussed below.

### Image Acquisition

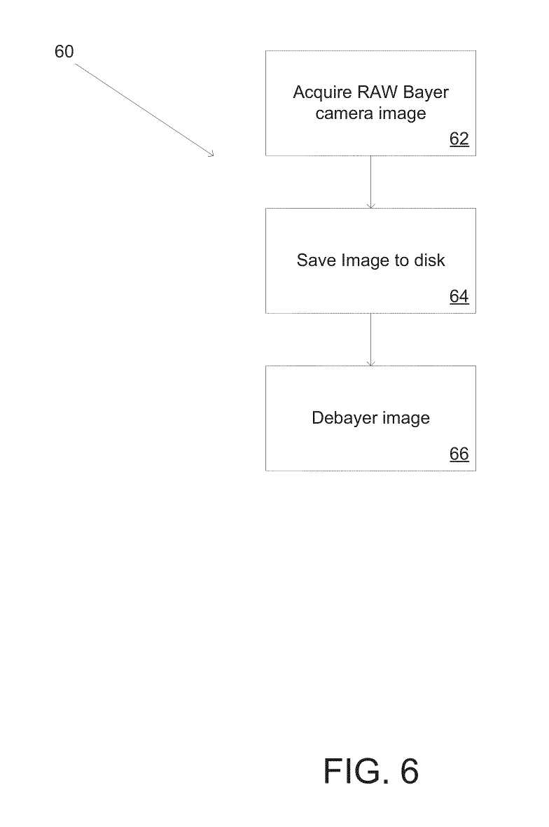

A process for acquiring an image using a digital camera in accordance with an embodiment of the invention is shown in FIG. 6. The acquisition process 60 includes acquiring (62) an image (in the illustrated embodiment a RAW Bayer camera image), saving (64) the image to a storage device such as a hard disk and debayering (66) the image to produce a captured color image that can also be stored. The capture of images is typically coordinated using a capture software module that interfaces with the endoscope camera hardware in order to pull captured images from the detector into a format that can be manipulated by the computer. In several embodiments, the capture module includes USB support, automatic white balancing, adjustment of image capture parameters (exposure time, frame rate and gain), and functionality for saving an image series to a disk. In many embodiments, the debayering operation is spatially variant interpolation. The interpolation operation can use nearest neighbor, linear or cubic interpolation algorithms. Other more advanced interpolation algorithms can also be used, such as the algorithm described in the IEEE ICASSP 2004 conference paper titled “High-Quality Linear Interpolation for Demosaicking of Bayer-Patterned Color Images” by Henrique S. Malvar, Li-wei He, and Ross Cutler, the disclosure of which is incorporated herein by reference in its entirety.

While use of Bayer filters within digital cameras is common, embodiments of the invention are equally applicable for use with images captured using a CGYM filter, an RGBE filter, vertically layered sensors and systems that use three separate sensors (such as 3 CCD cameras). In each embodiment, processes are performed to obtain an image. Images captured using appropriate cameras in accordance with embodiments of the invention are not limited to color images captured at visible light wavelengths. Embodiments of the invention can use cameras that capture monochrome images, thermal images, and/or other types of images that can be captured using an endoscope.

### Dewarping Captured Images

The image data acquired from the endoscope is typically distorted due to the optics of the endoscope (see for example FIGS. 3b and 4b). The specific distortions depend upon the nature of the endoscope. In a number of embodiments, the optics are radially symmetrical and images captured by the endoscope can be dewarped by applying a one dimensional dewarping function to a polar coordinate system and then mapping each point to a Euclidian coordinate system. Applying the one dimensional dewarping function involves locating the optical center of the image. Various algorithms can be used to locate the optical center of an endoscopic image, several of which are discussed below.

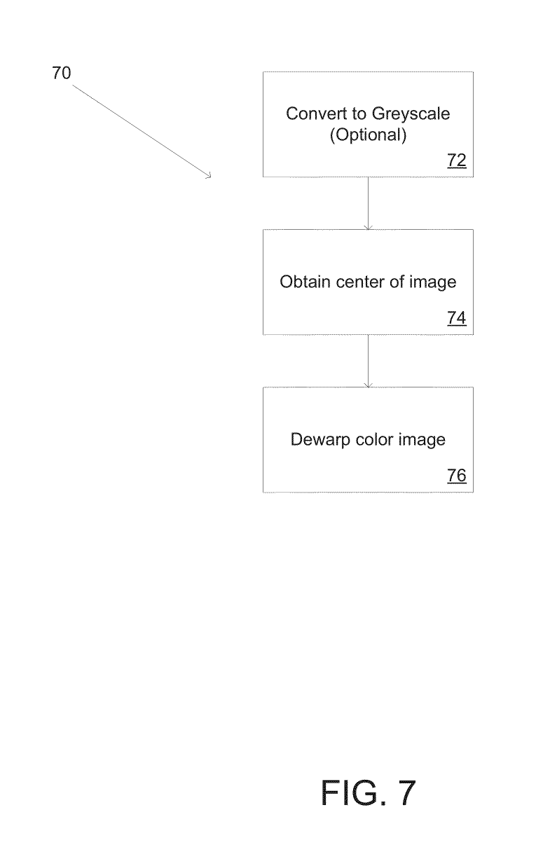

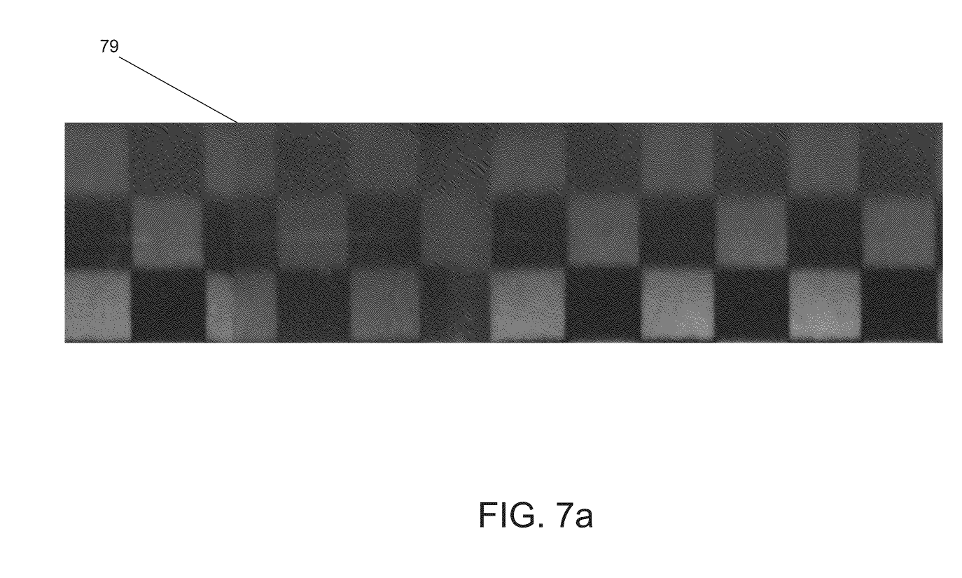

An efficient process for dewarping a color image in accordance with an embodiment of the invention is shown in FIG. 7. The process 70 includes converting (72) the color image to grayscale, obtaining (74) the optical center of the image and using the optical center to dewarp (76) the color image by mapping the image data from a radial non-uniformly sampled structure to a rectangular uniformly sampled structure. The conversion of the image to grayscale can reduce the amount of data that is processed while finding the location (74) of the optical center of the image. In embodiments that generate maps from endoscope images in real time, the reduced processing associated with use of grayscale information for some image processing steps can decrease system latency. The optical center of a grayscale image can be used to dewarp the color image from which the grayscale image was derived. When additional processing power is available, a real time system need not perform the grayscale conversion when locating the optical center of the color image. Similar processes can be used without the grayscale transformations for dewarping images that are not color images.

In many embodiments, finding the exact optical center of an image can be important to the mosaicking process. The exact optical center can involve location of a point (e.g. an endoscope including radially symetric optics), a center line (e.g. an endoscope with duck bill optics), or another reference dependent upon the nature of the endoscope. If the center location is off by a few pixels, the likelihood that the dewarping process will produce an accurate image diminishes. In several embodiments, the process uses a template matching system based on inherent internal reflections of the endoscope tip to locate the optical center. In a number of embodiments, the template is dependent upon the tip geometry, tip material properties and lighting conditions of the image acquisition. In several embodiments, fiducial markings are added to the endoscope tip that aid in center localization and improve the robustness of the center tracking process. In several embodiments, internal reflections create rings of brightness in the images captured by the endoscope and a template can be selected that uses these rings during center location. In other embodiments, fiducial markings are placed on the tip of the endoscope or within the optic system of the endoscope so that the fiducial markings appear in the images captured by the endoscope.

### Center Location Using Template Matching

In several embodiments, the optical center of an image is located by comparing the captured image to a template under various two-dimensional shifts. The similarity between the shifted template and the image can be estimated using the sum of absolute difference (SAD) between the template and input image. The center of the captured image can be located by finding the translation of the template that produces the smallest SAD. If the template image has width=w and height=h then the SAD for center location (cₓ,c_{y}) can be expressed as:

$$SAD ( {c}_{x} , {c}_{y} ) = {\sum }_{y = 0}^{y = h} {\sum }_{x = 0}^{x = w} abs ( \begin{matrix}template\_image ( x , y ) - \\ input\_image ( x + cx - w , y + cy - h )\end{matrix} )$$

The SAD is computed for all potential center-locations within a search window, and the location of the center of the template is determined to be the center location of the displaced grayscale captured image that gives rise to the smallest SAD. In many embodiments, including embodiments where fiducials are used to locate the image center, the center of template need not necessarily correspond to the center of the image. For example, the center of the template can define the location of the center of the image relative to one or more located fiducials. The search window is a defined area of pixels in which the center of the optical image is believed to be located. The size of the search window is typically chosen to maximize the likelihood that the optical center is located within the search window and to limit the amount of processing performed to locate the optical center.

In a number of embodiments, the search window employed in performing template matching can vary. When template matching is performed in what can be considered a full search mode, the SAD is calculated with a large window within the center of the captured image. In a number of embodiments, the search window is located within the central 128×128 pixels of the captured image. Once the center of the image has been located, then template matching can be performed in what can be considered a tracking mode. Template matching in tracking mode involves utilizing a narrower search window centered on the previous frame's center location. In a number of embodiments, the center tracking process can include multiple iterations to refine center detection. In several embodiments, center location can be repeated on an image in tracking mode in response to a full search being performed on a subsequently captured image. In tracking mode, many embodiments use a search window that is +/−12 pixels vertically and horizontally from the previous frame's center location. While in tracking-mode, if the center-location is found to be outside the center 128×128 pixel region, then it is possible that the center tracking operation has mistakenly lost the center-location. Therefore, the next frame processed can be processed in full-search mode to obtain a renewed estimate of the center-location. In many embodiments, the renewed estimate can be used to track backward through previously captured images in an attempt to correct for errors in previously dewarped images due to errors in the location of the center of the image. In many embodiments, converting an image to grayscale reduces processing latency during the performance of the center location processes.

### Dewarping Capture Images

In many embodiments, dewarping is performed by obtaining a dewarping transformation using a known image. The known image is captured using the endoscope and the transformation required to obtain the known image is determined. The transformation can then be used to dewarp other images captured by the endoscope. In embodiments where the endoscope has radially symmetric optics, a radially symmetric transformation can be used following the location of the center of the captured image.

An example of an image dewarped in accordance with an embodiment of the invention is shown in FIG. 7a. The dewarped image is obtained using a dewarping transformation applied to the image shown in FIG. 3b. In the dewarped image 79, the grid pattern on the interior surface of the lumen appears as even squares. In several embodiments, an interior surface including a grid pattern is used to determine an appropriate dewarping transformation. In many embodiments, other techniques for determining an appropriate dewarping transformation are used.

Due to distortions introduced by the optics of many endoscopes in accordance with embodiments of the invention, the resolution of the portions of the image that are closest to the base of the endoscope tip (i.e. the portion that is connected to the endoscope's illumination channel) is greatest. Resolution diminishes from the base to the tip and beyond the tip. In many embodiments, the dewarped image is cropped to discard portions of the image that do not possess sufficient resolution. Discarded information need not impact the final map of the interior surface of the cavity. A sufficiently high frame rate can ensure that the number of images captured as the endoscope moves through the cavity provides enough redundancy that a complete mosaic can be constructed from the highest resolution portions of each image. In many embodiments, the frame rate is set at 30 frames/sec. In other embodiments, the frame rate is determined based upon the speed with which the endoscope tip moves, the amount of each dewarped endoscope image that is discarded, and/or other requirements of the application. In this way, the best information from each image is preserved and mosaicked to construct a map of the interior surface of the cavity.

In a number of embodiments, other processes are used to improve the quality of the mosaic generated by the endoscope. The brightness of pixels in dewarped images captured by the endoscope can be adjusted to account for the different level of illumination intensity of different regions of the surface imaged by the endoscope. In many embodiments, pixels that do not contain information concerning the interior surface of the cavity due to known defects in the endoscope can be identified and compensated for by deleting the pixels, smoothing non-uniform illumination associated with the pixels, interpolating with adjacent pixels, and/or using image information from other captured images. In addition, blur detection can be performed to enable the selection of image information from less blurry images during the mosaicing process. In a number of embodiments, blur detection is performed by inspecting the motion vectors of pixel blocks from one image to the previously captured image. Regions of the image (i.e. pixel blocks) that possess motion vectors with a magnitude above a predetermined threshold can be considered blurry and, where alternative image information is available, can be discarded. In other embodiments, any variety of processes can be applied to captured images to select the best image information to combine into the mosaic.

### Combining Dewarped Images

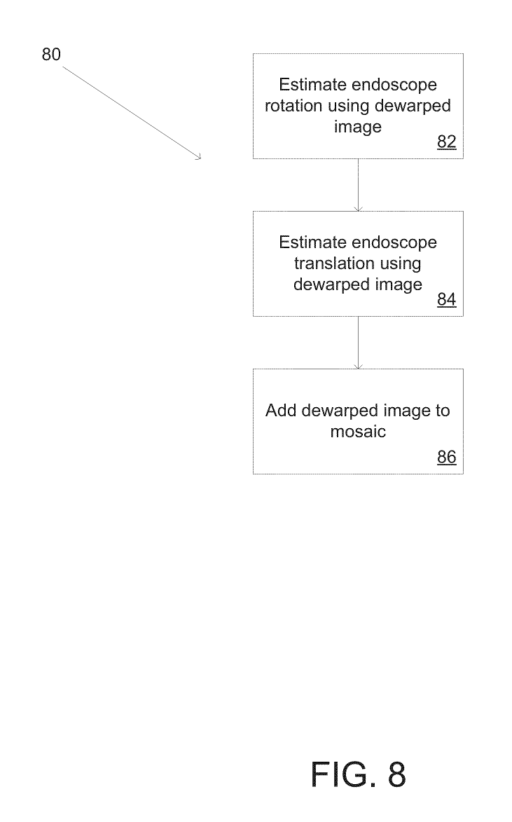

Once images have been dewarped, the images can be mosaicked together to increase the size of the map of the interior surface of the cavity being imaged by the endoscope. A process for mosaicking images in accordance with an embodiment of the invention is shown in FIG. 8. The process 80 includes estimating (82) the rotation of the tip of the endoscope, estimating (84) the translation of the endoscope tip, and then using the rotation and translation information to determine the area of the interior surface of the cavity captured within the image. The image information can then be added (86) to the map of the interior surface of the cavity.

Mosaicking Images from a Constrained Endoscope

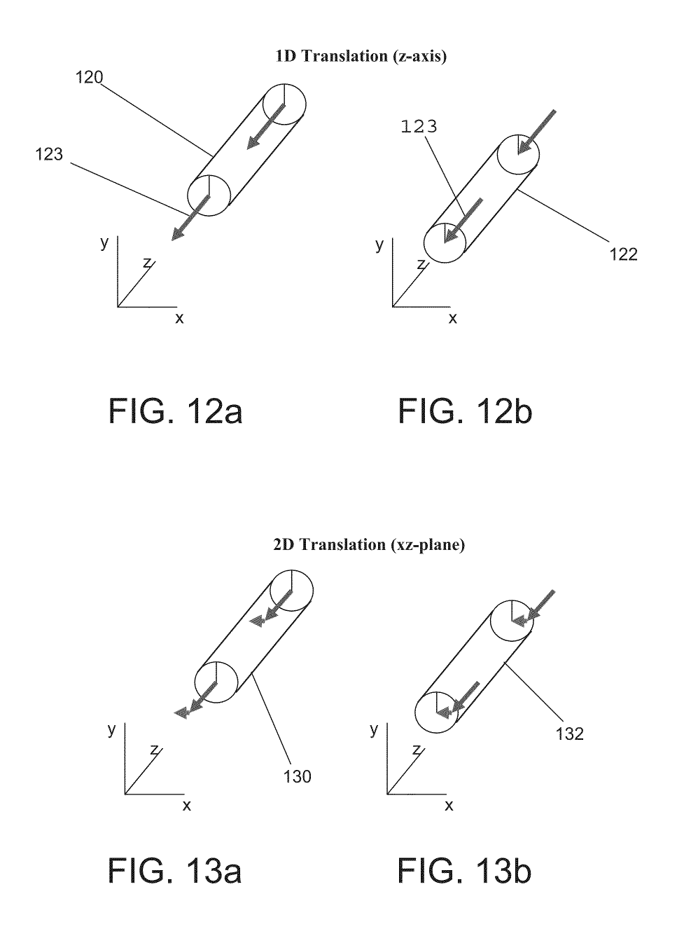

In many instances, the endoscope's motion is restricted to advancing within the cavity, retreating within the cavity and/or rotating around the endoscope's central axis within the cavity. Therefore, the motion estimation problem becomes estimation of rotation about a single axis and estimation of translation along a single axis. By performing motion tracking between each dewarped image and the previous image, the horizontal and vertical translation that occurs between images (where there is significant redundancy, registration can be performed across multiple images) can be used to estimate rotation and translation respectively. An example of rotation around the central axis of an endoscope is shown in FIGS. 11a and 11b and an example of one dimensional translation is shown in FIGS. 12a and 12b.

The manner in which translation of dewarped images can be estimated is similar to the template matching process with the exception that two sequentially captured images are compared. Due to the fair degree of local texture present in tissue such as the endometrial lining of a uterus, motion estimation can be performed by adapting efficient tracking algorithms such as the sum-of-squared distances efficient tracking process described in the paper Hager, G. D. and Belhumeur, P. N., 1998, Efficient Region Tracking with Parametric Models of Geometry and Illumination, IEEE Transactions on Pattern Analysis and Machine Intelligence, 20(10), p. 1025-1039. The process involves defining a difference function obtained by translating one of the pair of images formed by the time t image and the time t−1 image by an amount (u,v):

F(u,v)=Iₜ₋₁(x, y)−Iₜ(x+u, y+v)

The motion vector of the endoscope can be estimated by locating the value of the motion vector (u, v) at which the square of F(u, v) is a minimum. In many embodiments, the process of estimating the motion vector involves linearizing the square of the difference function by taking a first-order Taylor series expansion and then iterating until the minimum value for the comparison function is obtained. The Taylor series expansion is as follows:

$${( u , v )}^{*} = arg {min}_{( u , v )} {\sum }_{Λ} {\vert F ( 0 , 0 ) - ∇ F ( 0 , 0 ) ( \begin{matrix}u \\ v\end{matrix} ) \vert}^{2}$$

∇F is the Jacobian matrix of F and can be expanded as follows:

$${( u , v )}^{*} = arg {min}_{( u , v )} {\sum }_{Λ} {\vert {I}_{t - 1} ( x , y ) - {I}_{t} ( x , y ) - \frac{\partial }{\partial x} {I}_{t} ( x , y ) u - \frac{\partial }{\partial v} {I}_{t} ( x , y ) v \vert}^{2}$$

The above function is linear in parameters. Differentiating and equating to 0 gives a standard linear least-squares problem:

$$⌊ \begin{matrix}\frac{\partial }{\partial x} {I}_{t} ( 0 , 0 ) & \frac{\partial }{\partial y} {I}_{t} ( 0 , 0 ) \\ \frac{\partial }{\partial x} {I}_{t} ( 0 , 1 ) & \frac{\partial }{\partial y} {I}_{t} ( 0 , 1 ) \\ ⋮ & ⋮ \\ \frac{\partial }{\partial x} {I}_{t} ( m , n ) & \frac{\partial }{\partial y} {I}_{t} ( m , n )\end{matrix} ⌋ [ \begin{matrix}u \\ v\end{matrix} ] = [ \begin{matrix}{I}_{t - 1} ( 0 , 0 ) - {I}_{t} ( 0 , 0 ) \\ {I}_{t - 1} ( 0 , 1 ) - {I}_{t} ( 0 , 1 ) \\ ⋮ \\ {I}_{t - 1} ( m , n ) - {I}_{t} ( m , n )\end{matrix} ]$$

The solution to the above linear system is iterated until convergence. Using the negative spatial derivatives of the time t−1 image provides computation advantages over using the spatial derivatives of the time t image, because each iteration involves warping the time t image by the translation estimates from the previous iteration. Taking the derivatives of the translated time t image is an unnecessary computational cost on top of the spatial warp.

In addition to the iteration described above, many embodiments of the invention build a Gaussian image pyramid and apply the iterations to each image in the Gaussian image pyramid. A Gaussian image pyramid is a hierarchy of images derived from the original image where each image in the hierarchy is lower resolution than the image below it in the hierarchy (typically half the resolution). The Gaussian image pyramid serves two purposes:
1. The basin of attraction (the amount of motion that can be reliably estimated) is limited to the information provided by the local image gradients. An image pyramid extends this basin to avoid local minima.
2. The image pyramid also smoothes data reducing noise artifacts and produces more reliable image gradient calculations.

An estimated motion vector is obtained for each image in the Gaussian pyramid starting with the image having the lowest resolution. Once an estimated motion vector is obtained, the estimated motion vector is used as a starting point for the iterations used to obtain an estimate of the motion vector using the image in the Gaussian pyramids having the next highest resolution. The estimated motion vector of the highest resolution images in the Gaussian pyramids (i.e. the time t image and the time t−1 image) is the final estimate of the motion vector. In other embodiments, the motion vector is calculated in conjunction with a residual error calculation and multiple passes are made over the data set to estimate motion vectors in a way that attempts to minimize residual error. Over time, the motion vectors provide information concerning motion of the endoscope within the cavity. In a number of embodiments, thresholds are established with respect to aspects of the motion such as speed and rotation that can trigger alarms to warn the operator that the motion is likely to impact the quality of the captured images and the resulting mosaics of the interior surface of the cavity. In a number of embodiments, additional thresholds are defined that trigger alerts that warn the operator of the potential for the motion to cause discomfort or harm to the patient.

Mosaicking Images from Within a Collapsed Cavity

When an image is captured within a cavity, the image can contain segments of various walls of the cavity. In a number of embodiments, the motion estimation process includes identifying segments of the image corresponding to different surfaces of the cavity. The segments can be identified from the dewarped image or from the captured image. Once the segments have been identified, motion estimation can be performed on each segment and then the segments can be added to mosaics for each surface as appropriate.

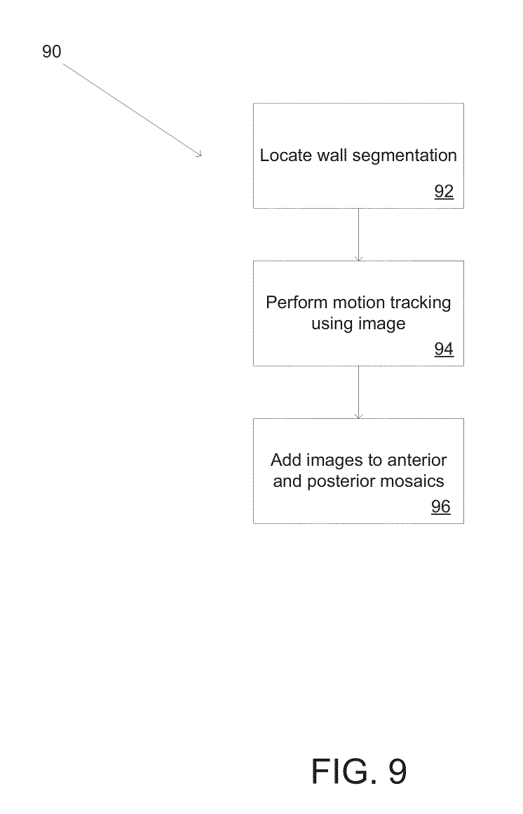

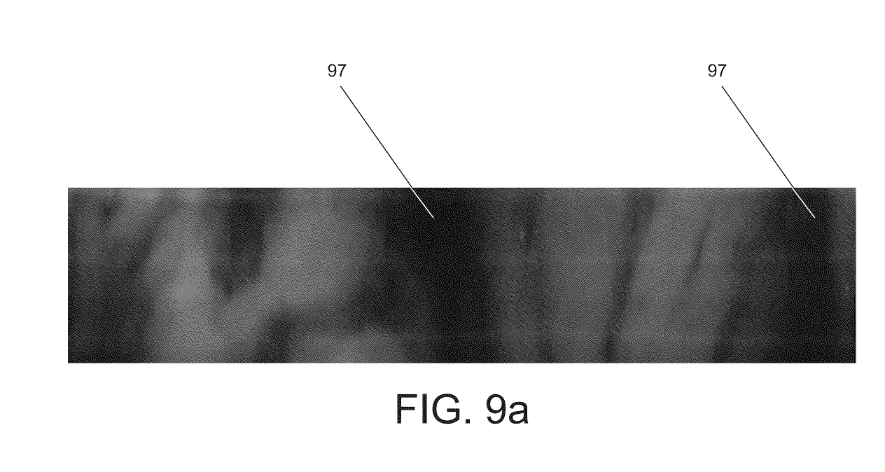

A process for identifying first and second wall segments from an endoscope image of a collapsed cavity and adding the segments to mosaics of the walls of the cavity is shown in FIG. 9. The process 90 includes locating (92) wall segments, performing (94) motion tracking for each segment, and adding (96) the segments to the anterior and posterior mosaics. In many embodiments, processing latency is reduced by performing motion tracking using grayscale versions of each segment of the dewarped images.

### Wall Segmentation

Collapsed cavities or systems of cavities can contain two or more surfaces. In the case of a uterus, the two surfaces are the anterior and posterior walls of the endometrium. An image of the anterior and posterior walls of the endometrium of a uterus captured using an endoscope in accordance with an embodiment of the invention is illustrated in FIG. 4b. A dewarped image generated using the image shown in FIG. 4b is illustrated in FIG. 9a. The two surfaces of the endometrium coapted around the tip of the endoscope are delineated by two dark bands 97 in the dewarped image. When an endoscope captures an image using an omni-directional lens around which a collapsed cavity is coapted, the captured image can be segmented to build mosaics of the walls of the cavity. In a number of embodiments, segmentation is performed using dewarped image data.

In embodiments where the dewarped captured image is a rectangular image, the segmentation process can involve searching for dark lines in the image data assuming that these are segment boundaries. A problem that can be encountered when locating segment boundaries by searching for dark lines is that the segment boundaries are not fixed and can rotate to an extent that the process incorrectly swaps the surfaces of the cavity with which each segment corresponds.

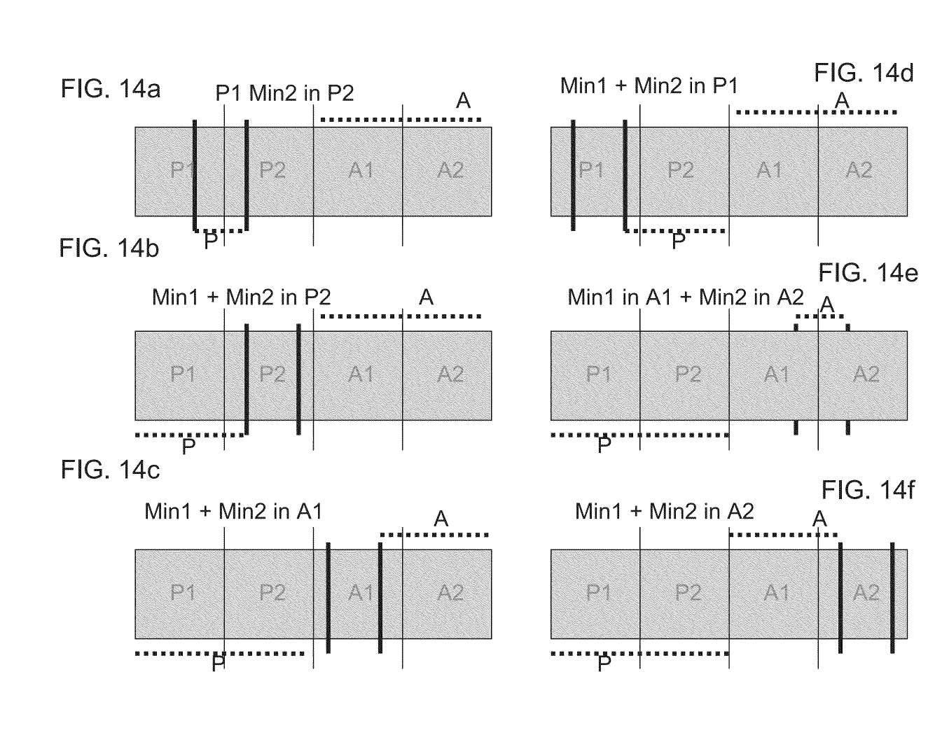

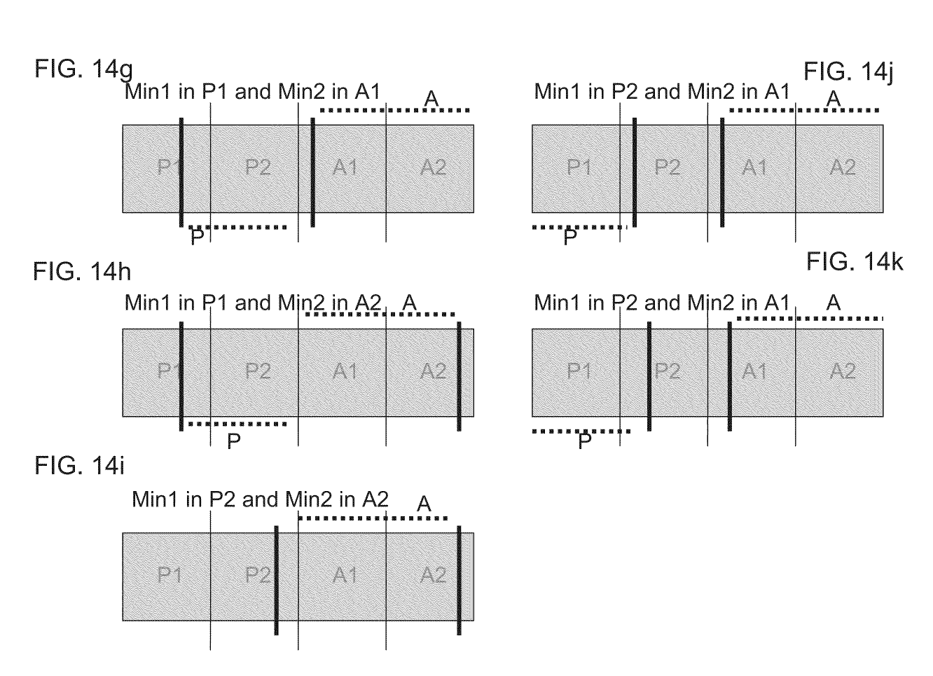

In several embodiments, the potential for swapping is addressed by assuming there is little axial rotation. Such an approach assumes that the first segment is always in contact with the 180-degree point and the second segment is always in contact with the 0-degree point. The effect of the assumption is to confine the first segment of the image and the second segment of the image to each respective half of the dewarped image. The portion of each respective half that is selected is the first segment and the second segment depends upon the location of the two darkest columns of the image. In one embodiment, the dewarped image is split into 4 sections (P1, P2, A1, A2) according to the portions corresponding to an angle around the circumference of the unwarped image of 0, 90, 180, and 270 degrees. The manner in which the first and second segments of the image are selected is based upon the location of the two darkest regions of the image is illustrated in FIGS. 14a-14k.

In other embodiments, a variety of alternative techniques can be used for identifying segments of images in a manner that prevents segments of the images being incorrectly associated with different surfaces of the cavity as the endoscope rotates. For example, the motion vectors of blocks of pixels within different segments of a captured image corresponding to different walls are typically parallel and moving in different directions. Therefore, boundaries at which two sets of parallel motion vectors meet or depart from can be used to identify segments of the captured image corresponding to different surfaces of the cavity. In other embodiments, rotation detection, feature detection, motion vectors, hysteresis and/or predication (interpolation or extrapolation) can be used to locate and or improve the location of segments of the image corresponding to different surfaces of an imaged cavity. Once the segments of the image have been identified, the segments can be further processed using motion tracking and mosaicking to add the segments to the map of each surface of the cavity.

When the endoscope is moved into the extreme left or right side of a cavity such as the uterus where first and second surfaces “meet” such that there is only one dark band, segmentation based on locating two dark bands may fail and several techniques may be used to identify and resolve this situation: when probe movement stops and reverses over a series of frames and during a portion of these frames there is only one very dark band then it can be assumed that the “wall” has been reached and the dividing line between first and second surfaces can be defined as the point (line) 180 degrees away from the one very dark band; or the point (line) which was detected in the last frame in which there were two very dark bands; or the point (line) which is an average or other combination of the point (line) calculated from the frame(s) just before and the frame(s) just after the frame(s) in which only one very dark band appears; or hysteresis or memory may be applied to the line position such that the point (line) between segments is only allowed to change by a certain number of pixels per frame and if no very dark band is detected then the points (lines) between segments will be kept in a constant location until future frames exhibit one or more dark bands from which a new location may be detected.

### Motion Estimation Within a Collapsed Cavity

The constraints that can be assumed when performing motion estimation within a cavity depend upon the nature of the cavity and features of the endoscope including the frame rate of the endoscope camera. In several embodiments, an endoscope is used to image the endometrial lining of an uninsufflated uterus. When a high frame rate (e.g. 30 frames/sec) is used and the insertion and withdrawal speed of the endoscope is slow, most of the frame-to-frame motion of the captured images of the endometrial lining is two-dimensional translation with a small amount of rotation (0.5° or less). In other applications, embodiments of the invention assume greater or more restricted freedom of movement and/or apply a frame rate appropriate to the specific type of endoscope being used and the constraints of the specific application.

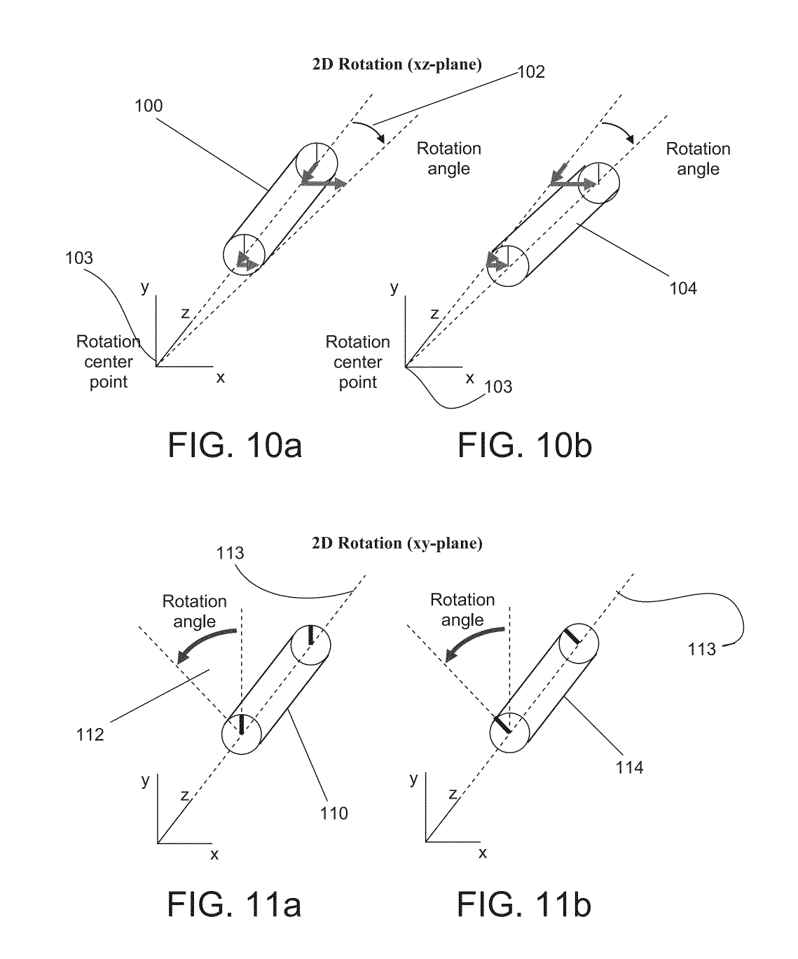

The two dimensional rotation of the tip of an endoscope about a rotation center point is illustrated in FIGS. 10a and 10b. The tip of the endoscope is represented as a cylinder (a reflection of the region of the captured image that is typically retained following dewarping) and the tip is rotated from a first position 100 through an angle 102 about a rotation center point 103 to a second position 104. In many embodiments, the rotation is estimated. In other embodiments, the rotation is assumed small and mosaicking is simply performed by segmenting the warped image and estimating two dimensional motion using motion tracking.

Rotation of the tip of the endoscope around the central axis of the endoscope is shown in FIGS. 11a and 11b. The tip of the endoscope rotates from a first position 110 through an angle of rotation 112 around the central axis of the endoscope 113 to a second position 114. In many embodiments, the rotation is estimated. In other embodiments, the rotation is assumed small and mosaicking is simply performed by segmenting the warped image and estimating two dimensional motion using motion tracking.

One-dimensional translation of the tip of an endoscope within a cavity in a direction along the central axis of the endoscope is shown in FIGS. 12a and 12b. The endoscope tip is translated from a first position 120 to a second position 122 along the central axis 123 of the endoscope tip. Two-dimensional translation of the tip of an endoscope within a cavity is shown in FIGS. 13a and 13b. In the illustrated embodiment, the translation includes a component along the central axis of the endoscope and a component perpendicular to the central axis of the endoscope. The two dimensional plane of the translation is typically defined by the interior surface of the cavity. The translation is from a first position 130 to a second position 132. Motion tracking techniques similar to those outlined above with respect to an endoscope that has restricted freedom of movement can be applied to segmented images to perform two dimensional motion tracking.

### Mosaicking Images

A mosaic can be thought of as a composite representation where information from one or more images can be combined. In a number of embodiments, mosaicking is performed by registering each dewarped image or image segment in a sequence to the previous dewarped image or image segment. Registration is a term used to describe aligning two images with respect to each other based upon information that is common to both images. Sequential registration maintains local consistency, however, the resulting mosaic is susceptible to global inconsistencies due to accumulation of error in frame-to-frame tracking (i.e. drift). A variety of techniques can be used for combining a dewarped image or image segment with a mosaic. In a number of embodiments, alpha-blending/compositing methods are used to combine overlapping pixel data. These techniques can include averaging all samples, averaging a selected portion of the available samples and/or using pixels with the best image quality near the proximal end of the optical window (i.e. the portion of each image that has the highest resolution). In a number of embodiments, the highest quality pixels are used regardless of location of the pixels within each image. In several embodiments, signal processing algorithms are applied to overlapping portions of the images in order to perform resolution enhancement (i.e. creating a hyper-resolution mosaic). In other embodiments, other techniques including quality metrics such as blur detection can be used to combine images in accordance with the constraints of a particular application.

In a number of embodiments, multiple passes are used to improve the registration between the mosaicked images. When images are registered sequentially (i.e. each image is registered with respect to the previous image in the sequence), errors accumulate over time. Additional passes can be used to reduce errors by identifying images that are of the same region of the surface being imaged and registering these images with respect to each other. In a number of embodiments, a comparison is performed to determine whether features present in one image are present in any of the other captured images of the surface. To the extent that the mosaic does not correctly register these images, the images can be reregistered with respect to each other. In many embodiments, the re-registration provides information concerning other images adjacent to the re-registered images in the mosaic that are likely to be of the same portion of the imaged surface. These adjacent images can also be re-registered with respect to each other. In this way, the errors accumulated in the pass can be reduced by successive passes and re-registration of images of the same portion of the imaged surface. In other embodiments, a variety of other techniques can be used to improve the accuracy of the registration of the mosaicked images by identifying images corresponding to multiple passes over the same region of the imaged surface by the endoscope. For example, the second pass can search for images separated by a predetermined period of time that are within a predetermined distance of each other. The located images can be compared and re-registered to align the images with respect to each other. In a number of embodiments, a sequential process is applied to produce a real time image(s) and a multiple pass process can be applied to produce a more precise image(s).

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method of imaging the interior surface of a cavity, comprising:
capturing images using an endoscope, where the optics of the endoscope are radially symmetrical;
locating the optical center of each of the captured images;
dewarping each of the captured images by mapping the image from polar coordinates centered on the optical center of the image to rectangular coordinates;
discarding portions of each dewarped image to create high clarity dewarped images;
estimating the motion of the endoscope with respect to the interior surface of the cavity that occurred between successive high clarity dewarped images;
registering the high clarity dewarped images with respect to each other using the estimates of motion; and
combining the registered high clarity dewarped images to create at least one mosaic.

2. The method of claim 1, wherein the optical center is located by comparing the captured image to a template image.

3. The method of claim 2, wherein the optical center is located by locating the translation between the captured image and the template image that results in the smallest sum of absolute differences.

4. The method of claim 3, wherein:
a large range of possible translations are considered in locating the optical center of a first captured image; and
a small range of possible translations are considered relative to the location of the optical center of the first captured image in locating the optical center of a second captured image.

5. The method of claim 1, wherein the endoscope includes a tip through which images are captured and the tip includes fiducial markings that assist in the location of the optical center of captured images.

6. The method of claim 1, wherein discarding portions of each dewarped image that possess insufficient image clarity to create high clarity dewarped images comprises discarding a predetermined portion of each dewarped image.

7. The method of claim 1, wherein discarding portions of each dewarped image that possess insufficient image clarity to create high clarity dewarped images comprises:
performing blur detection on each image; and
discarding at least one region of the image possessing blurriness exceeding a predetermined threshold.

8. The method of claim 1, further comprising adjusting the brightness of pixels to account for variations in the illumination of the imaged surface.

9. The method of claim 1, further comprising compensating for pixels within the captured images that are the result of known defects in the endoscope.

10. The method of claim 1, wherein estimating the motion of the endoscope with respect to the interior surface of the cavity that occurred between successive high clarity dewarped images comprises determining a motion vector at which the square of the differences between successive captured images is a minimum.

11. The method of claim 10, wherein determining the motion vector at which the square of the differences between successive captured images is a minimum comprises:
creating a Gaussian pyramid for each image; and
using the motion vector at which the square of the differences between images of corresponding lower resolution in the Gaussian pyramids is a minimum to determine the motion vector at which the square of the differences between images of corresponding higher resolution images in the Gaussian pyramids is a minimum, until the motion vector at which the square of the differences between the two captured images is a minimum is determined.

12. The method of claim 1, further comprising:
performing multiple passes over the captured images to improve the accuracy of the estimation of the motion of the endoscope with respect to the interior surface of the cavity compared to the initial estimate determined by comparing successive high clarity dewarped images; and
performing multiple passes to register successive images with respect to each other to reduce registration errors accumulated during initial sequential registration.

13. The method of claim 1, wherein images are captured at a frame rate chosen so that the motion that occurs between the captured images is sufficiently small for the high clarity dewarped images to overlap.

14. The method of claim 1, wherein:
the captured images are color images;
the system generates mosaics in real time; and
processing latency is reduced by converting the captured images from color to grayscale and locating the optical center of the grayscale images.

15. The method of claim 1, wherein processing latency is reduced by dewarping grayscale images and performing motion estimation using grayscale images.

16. The method of claim 1, further comprising performing image segmentation to identify segments of the image corresponding to different surfaces of the cavity and combining the image segments corresponding to different surfaces of the cavity to form separate mosaics of each of the different surfaces of the cavity.

17. The method of claim 16, wherein performing image segmentation further comprises locating at least the two darkest columns in the high clarity dewarped images.

18. The method of claim 17, further comprising limiting the rotation of the endoscope and assuming the two darkest columns are constrained to be located within defined regions of the high clarity dewarped images.

19. The method of claim 18, wherein the defined regions correspond to the two halves of the field of view of the endoscope.

20. The method of claim 16, comprising using boundaries between groups of aligned motion vectors of blocks of pixels within the image to identify segments of the image corresponding to different surfaces of the cavity.
